(19) United States
(12) Patent Application Publication
(10) Pub. No.: US 2021/0322978 A1
(43) Pub. Date: Oct. 21, 2021

(54) FLOW PASSAGE DEVICE FOR BIOLOGICAL COMPONENT EXAMINATION AND BIOLOGICAL COMPONENT EXAMINATION SYSTEM

(71) Applicant: H.U. Group Research Institute G.K., Tokyo (JP)

(72) Inventors: Tatsuki Matsuno, Tokyo (JP); Takeyuki Kotaka, Tokyo (JP); Wataru Tonomura, Tokyo (JP)

(21) Appl. No.: 17/163,441

(22) Filed: Jan. 30, 2021

Related U.S. Application Data
(63) Continuation-in-part of application No. PCT/JP2019/043719, filed on Nov. 7, 2019.

(30) Foreign Application Priority Data
Nov. 12, 2018 (JP) 2018-212277

(51) Int. Cl. B01L 3/00

(52) U.S. Cl. CPC B01L 3/502715; B01L 2300/0809; B01L 2300/04; B01L 2200/027

(57) ABSTRACT

A flow path device 20 is a biological component test flow path device for storing a specimen used to test a biological component in a specimen separated from a living body and includes: a main body portion 30; at least one or more flow paths 40 which are provided inside the main body portion 30 so that at least one or more inlet open ends 41 and at least one or more outlet open ends 42 in a plurality of different open ends of the at least one or more flow paths 40 overlap each other or are adjacent to each other and are exposed to the outside of the main body portion 30; and a lid portion 50 which opens and closes at least one or more inlet open ends 41 and at least one or more outlet open ends 42 together.

### CROSS REFERENCE TO RELATED APPLICATIONS

The present patent application is a continuation-in-part from the international patent application PCT/JP2019/043719 filed on Nov. 7, 2019 and now published as WO 2020/100720, which claims priority from the Japanese patent application No. 2018-212277 filed on Nov. 12, 2018. The disclosure of each of the above-identified patent applications is incorporated herein by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### TECHNICAL FIELD

The present invention relates to a biological component test flow path device and a biological component test system.

### BACKGROUND ART

Conventionally, in biological component tests, particularly nucleic acid detection, since nucleic acid amplification tests are performed in a short time, there is known a method of increasing the heating efficiency of a test liquid by storing a test liquid (a mixed solution of a reagent and a specimen) in a device including a micro flow path portion and reacting the test liquid, for example, as described in Patent Document 1. Since the device generally has a thin flat plate-shaped structure, the device has an advantage that heat from an adjacent heating portion is easily transferred to the test liquid contained therein and the temperature of the test liquid can be adjusted in a short time.

### CITATION LIST

### Patent Document

Patent Document 1: Laid-open Patent Application Publication in Japan No. 2017-23141

### SUMMARY OF THE INVENTION

### Technical Problem

In the above-described micro flow path device, since a small amount of the test liquid is introduced thereinto and is heated, it is required to seal the flow path after introducing the test liquid thereinto in order to prevent the test liquid from evaporating. Therefore, it is necessary to block an inlet for introducing the test liquid into the device and an outlet for extracting an internal gas. In the related art, a mechanism for blocking the inlet and the outlet by a seal has been provided. However, when the seal is automatically attached by a machine, it requires very fine control. For this reason, since this is difficult, the seal is attached manually in general. Therefore, there is room for improvement from the viewpoint of workability.

It is an object of the present invention to solve the problems of the above mentioned prior arts.

### Means to Solve Problem

One aspect of the present invention provides a biological component test flow path device for storing a substance used to test a biological component in a specimen separated from a living body, the biological component test flow path device comprises: a main body portion; at least one or more flow paths which are provided inside the main body portion and are provided so that at least one or more inlet open ends and at least one or more outlet open ends in a plurality of different open ends of the at least one or more flow paths overlap each other or are adjacent to each other and are exposed to the outside of the main body portion; and a lid portion which opens and closes the at least one or more inlet open ends and the at least one or more outlet open ends together.

Another aspect of the present invention provides a biological component test system comprises: the biological component test flow path device according to any one of claims 1 to 4.

### BRIEF DESCRIPTION OF THE DRAWINGS

FIGS. 1A, 1B are diagrams illustrating a biological component test tip according to an embodiment of the present invention, where FIG. 1A is a front view and FIG. 1B is a bottom view.

FIGS. 2A, 2B are diagrams illustrating a flow path device, where FIG. 2A is a plan view and FIG. 2B is a front view.

FIGS. 3A, 3B, 3C are diagrams illustrating the flow path device in a state in which a lid portion is detached, where FIG. 3A is a plan view, FIG. 3B is a cross-sectional view taken along line A-A of FIG. 3A, and FIG. 3C is a cross-sectional view taken along line B-B of FIG. 3A.

FIG. 4 is an enlarged view of the periphery of the tip in a state in which the tip of FIG. 1 is inserted into the flow path device of FIGS. 3A, 3B, 3C (a part of them is not illustrated).

FIG. 5 is a diagram illustrating a modified example of the lid portion and is a diagram illustrating an area corresponding to FIG. 2B.

FIG. 6 is a diagram illustrating a modified example of the tip and is a diagram illustrating an area corresponding to FIG. 1A.

FIGS. 7A, 7B are diagrams illustrating a modified example of a shape maintaining portion, where FIG. 7A is a diagram illustrating an area corresponding to FIG. 1A and FIG. 7B is a diagram illustrating an area corresponding to FIG. 1B.

FIGS. 8A, 8B are diagrams illustrating a modified example of the shape maintaining portion, where FIG. 8A is a diagram illustrating an area corresponding to FIG. 1A and FIG. 8B is a diagram illustrating an area corresponding to FIG. 1B.

FIGS. 9A, 9B are diagrams illustrating a modified example of the shape maintaining portion, where FIG. 9A is a diagram illustrating an area corresponding to FIG. 1A and FIG. 9B is a diagram illustrating an area corresponding to FIG. 1B.

FIGS. 10A, 10B provide a front view illustrating a modified example of the shape maintaining portion, where FIG. 10A is a diagram illustrating a state before the tip is inserted and FIG. 10B is a diagram illustrating a state in which the tip is inserted.

FIGS. 11A, 11B provide a front view illustrating a modified example of the shape maintaining portion, where FIG. 11A is a diagram illustrating a state before the tip is inserted and FIG. 11B is a diagram illustrating a state in which the tip is inserted.

FIGS. 12A, 12B are diagrams illustrating modified examples of an inlet open end and an outlet open end, where FIG. 12A is a diagram illustrating an area corresponding to FIG. 2A and FIG. 12B is a diagram illustrating an area corresponding to FIG. 2B.

FIGS. 13A, 13B, 13C are diagrams illustrating modified examples of the inlet open end and the outlet open end, where FIG. 13A is a diagram illustrating an area corresponding to FIG. 2A, FIG. 13B is a diagram illustrating an area corresponding to FIG. 2B, and FIG. 13C is a cross-sectional view taken along line C-C of FIG. 13A (a part of them is not illustrated).

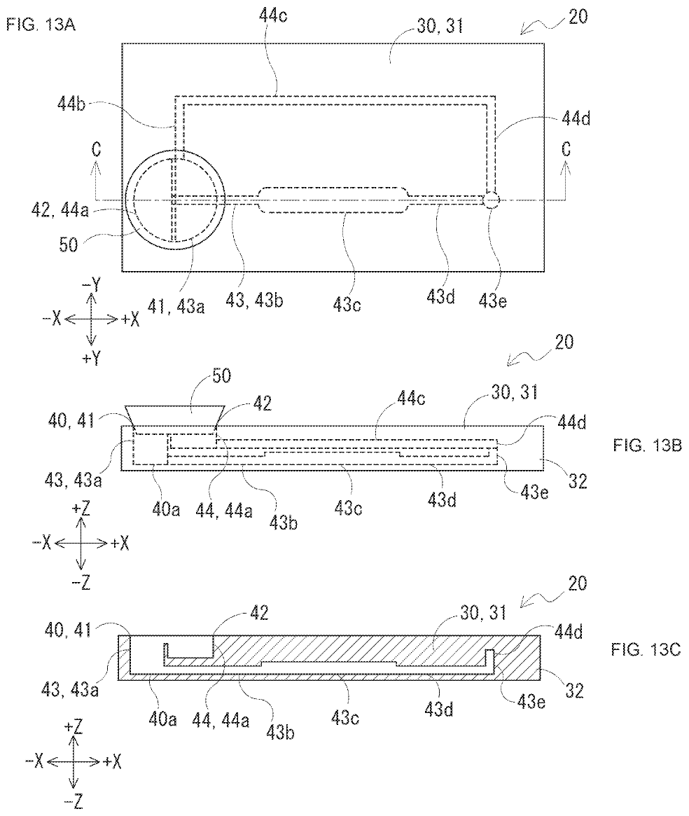

### MODE FOR CARRYING OUT THE INVENTION

Hereinafter, embodiments of a biological component test flow path device and a biological component test system according to the invention will be described in detail with reference to the accompanying drawings. First, [I] Basic concept of embodiment will be described, [II] Detailed content of embodiment will be described, and finally [III] Modified example of embodiment will be described. However, the present invention is not limited to the embodiment.

### [I] Basic Concept of Embodiment

First, the basic concept of the embodiment will be described. The embodiment schematically relates to a biological component test flow path device for storing a substance used for biological component test and a biological component test system.

Here, the “biological component test” means to detect and/or quantify a target component in a sample separated from a living body and corresponds to, for example, a biochemical test, a hematological test, a histological test, an immunological test, a gene detection test (nucleic acid amplification test, southern hybridization, etc.), sequence analysis (nucleic acid, protein, sugar chain, etc.), etc. Further, the type of “sample used for biological component test” is a sample separated from a living body and examples thereof include serum, plasma, whole blood, blood cell components, urine, stool, sputum, spinal fluid, oral mucosa, pharyngeal mucosa, intestinal mucosa, vaginal mucosa, and biopsy samples (for example, Fine needle aspiration (FNA) samples, intestinal samples, and liver samples). Alternatively, a treated sample obtained by treating these with an acid, an alkali, a protein denaturant, a surfactant, an oxidizing agent, a reducing agent, an enzyme, a dilution, filtration, extraction, heating, etc., or a combination thereof can be mentioned. The “test liquid” in the present invention may be the sample (including the treated sample) itself or may be a mixture of the sample and a solid, semi-solid, or liquid substance (for example, a specimen) other than the sample or a solid, semi-solid, or liquid substance (for example, a reagent such as a magnetic particle or a label, a cleaning liquid, and a solvent) other than a test target. Further, the “biological component test system” is a system that performs a biological component test on a test liquid discharged from a tip to be described below to the biological component test flow path device. Hereinafter, in the embodiment, a case will be described in which the biological component test system is a system used for nucleic acid amplification tests based on a real-time PCR method and the biological component test flow path device is a device that stores a treated sample having undergone DNA extraction processing, a reagent for real-time PCR (including DNA polymerase, dNTP, primer, fluorescently labeled probe, etc.), or a test liquid obtained by mixing these. Note that, the “real-time PCR method” is a type of PCR method for amplifying DNA or RNA by polymerase chain reaction (PCR) and is a method for monitoring and analyzing the amplified DNA or RNA in real time. For example, an intercalation method using a fluorescent substance or an electrochemical substance, a hybridization method using a fluorescently labeled probe, a turbidity detection method, an electric detection method, etc. are applicable, but in the embodiment, the hybridization method, particularly, a TaqMan (trademark) method will be described.

### [II] Detailed Content of Embodiment

Next, the detailed content of the embodiment will be described.

(Configuration)

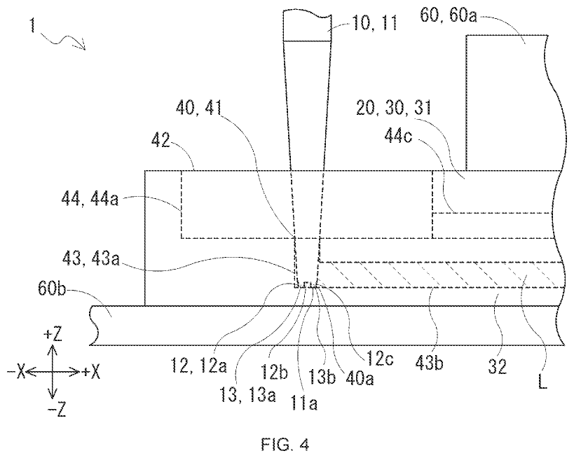

First, the configuration of the biological component test system that adopts the biological component test tip according to the embodiment will be described. The biological component test system 1 schematically shown in FIG. 4 includes a tip 10, a flow path device 20, a temperature controller 60, a discharge and suction member, an attachment and detachment portion, a detector, and a control unit. Specifically, when the discharge and suction member, the attachment and detachment portion, the temperature controller 60, and the detector are respectively and electrically connected to the control unit, it is possible to directly or indirectly perform the communication or power supply between the discharge and suction member, the attachment and detachment portion, the temperature controller 60, or the detector and the control unit.

(Configuration-Discharge and Suction Member)

The discharge and suction member has a shape capable of attaching the tip 10 thereto and is used to discharge and suck a test liquid L through the tip 10. This discharge and suction member is configured by using, for example, a known test dispensing device (for example, a dispensing device including a nozzle and a pump (not illustrated)) or the like and is provided in the vicinity of the flow path device 20.

(Configuration-Attachment and Detachment Portion)

The attachment and detachment portion is an attachment and detachment member attaching and detaching a lid portion 50 of the flow path device 20 to be described later to and from a main body portion 30 of the flow path device 20 to be described later. This attachment and detachment portion is configured by using, for example, a known test chuck mechanism (for example, a mobile chuck mechanism) or the like and is provided in the vicinity of the flow path device 20.

(Configuration-Detector)

The detector is for detecting a target component contained in the test liquid L stored in the flow path device 20. This detector is configured by using, for example, a known detection device (for example, a spectrofluorometer) and is provided in the vicinity of the flow path device 20.

(Configuration-Control Unit)

The control unit is a unit controlling each component of the biological component test system 1 and includes an operation unit, a communication unit, an output unit, a power supply unit, a controller, and a storage unit (all of them are not illustrated).

(Configuration-Control Unit-Operation Unit, Communication Unit, Output Unit, Power Supply Unit)

The communication unit is a communication section communicating with the discharge and suction member, the attachment and detachment portion, the temperature controller 60, or the detector. The output unit is an output section for outputting various information items based on the control of the control unit and is configured by using, for example, a known display section or audio output section. The power supply unit is a power supply section for supplying power supplied from a commercial power supply or a battery (for example, a battery, etc.) to each part of the control unit and supplying the power to the discharge and suction member, the attachment and detachment portion, the temperature controller 60, or the detector.

(Configuration-Control Unit-Controller)

The controller is a control section controlling each part of the control unit. Specifically, this controller is a computer that includes a CPU, various programs analyzed and executed on the CPU (including a basic control program such as an OS and an application program started on the OS and realizing a specific function), and an internal memory such as a RAM storing a program or various types of data.

(Configuration-Control Unit-Storage Unit)

The storage unit is a storage section storing a program and various types of data necessary for operating the control unit, is configured by using a known rewritable recording medium, and can use, for example, a non-volatile recording medium such as a flash memory.

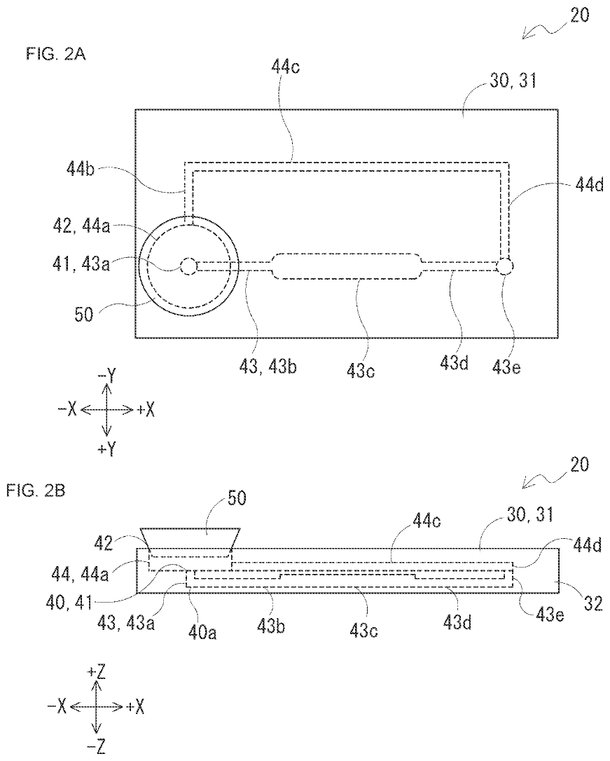

In the following description, the X direction of FIGS. 2A, 2B is referred to as the left and right direction of the flow path device (the −X direction is the left direction of the flow path device and the +X direction is the right direction of the flow path device), the Y direction is referred to as the front and rear direction of the flow path device (the +Y direction is the front direction of the flow path device and the −Y direction is the rear direction of the flow path device), and the Z direction is the up and down direction of the flow path device (the +Z direction is the up direction and the −Z direction is the down direction).

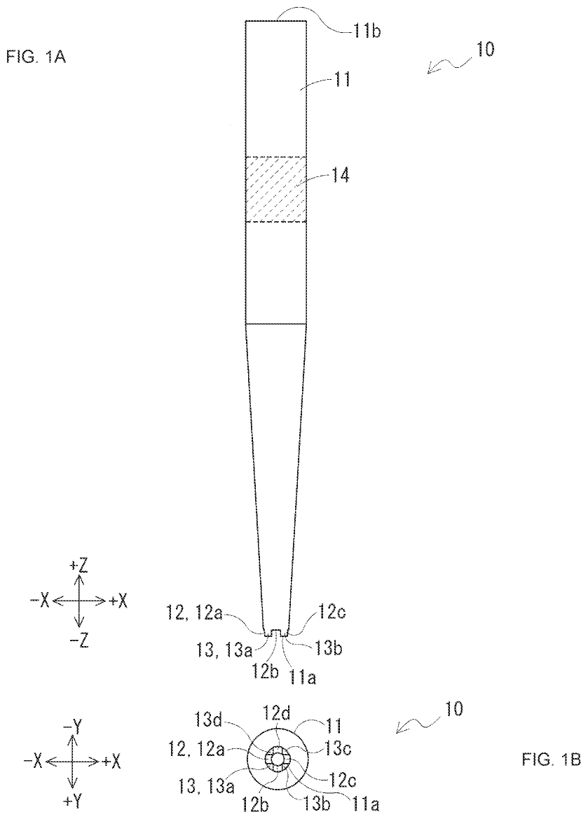

In the embodiment, the tip 10 includes, as illustrated in FIGS. 1A, 1B, and 4, a tip main body 11, opening portions 12a to 12d, and shape maintaining portions 13a to 13d. Additionally, the opening portions 12a to 12d will be simply and collectively referred to as the “opening portion 12” when it is not necessary to particularly distinguish them from each other and the shape maintaining portions 13a to 13d will be simply and collectively referred to as the “shape maintaining portion 13” when it is not necessary to particularly distinguish them from each other.

(Configuration-Detailed Configuration of Tip-Tip Main Body)

Returning to FIGS. 1A, 1B, the tip main body 11 is a basic structure of the tip 10. This tip main body 11 is formed as a cylindrical body (specifically, an elongated cylindrical body) and is provided so that the longitudinal direction of the tip main body 11 follows the up and down direction as illustrated in FIGS. 1A, 1B. Specifically, a lower open end 11a of the tip main body 11 is disposed so as to be in contact with or close to a bottom surface portion 40a of the flow path device 20 to be described later.

Further, the shape and size of the tip main body 11 are arbitrary, but are as below in the embodiment. That is, as illustrated in FIGS. 1A, 1B, and 4, the side surface shape of the tip main body 11 (particularly, a lower portion of the tip main body 11) is tapered so that the outer diameter of the tip main body 11 decreases toward the lower open end 11a. Further, the diameter of the upper open end 11b is set to a size that can be fitted to a nozzle of the discharge and suction member. Specifically, the diameter is substantially the same as the maximum outer diameter of the portion inserted through the tip main body 11 in the outer diameter of the nozzle of the discharge and suction member and is larger than the diameter of an inlet open end 41 of the flow path device 20 to be described later (or equal to or smaller than the diameter of the inlet open end 41). Further, the diameter of the lower open end 11a is set to a size that can be inserted through a first inlet open end side portion 43a of the flow path device 20 to be described later and can discharge a desired amount of the test liquid L. Specifically, the diameter is set to be smaller than the diameter of the inlet open end 41 of the flow path device 20 to be described later.

(Configuration-Detailed Configuration of Tip-Opening Portion)

Returning to FIGS. 1A, 1B, the opening portions 12a to 12d are used to allow the test liquid L to flow in and out from the side of the tip main body 11 when the test liquid L is discharged or sucked in a state in which the end portion on the discharge side or the suction side of the test liquid L in the end portion of the tip 10 is in contact with the bottom surface portion 40a of the flow path device 20 to be described later (hereinafter, referred to as a “contact state”). The opening portions 12a to 12d are provided to communicate with the lower open end 11a of the tip main body 11. Specifically, as illustrated in FIGS. 1A, 1B, and 4, a plurality of the opening portions are provided to be away from each other at the lower end portion of the tip main body 11 and the vicinity thereof.

Further, the shape and size of the opening portions 12a to 12d can be arbitrarily set so that the total discharge amount of the test liquid L of the opening portions 12a to 12d is substantially the same as the discharge amount of the test liquid L of the lower open end 11a, but are set as below in the embodiment. That is, as illustrated in FIGS. 1A, 1B, and 4, the side surface shape of the opening portions 12a to 12d is set to be substantially rectangular. However, the present invention is not limited thereto, but, for example, a polygonal shape such as a triangular shape or a semi-circular shape may be used. Further, the width of the opening portions 12a to 12d is set to be shorter than the outer diameter of the tip main body 11 and the height of the opening portions 12a to 12d (the length in the up and down direction) is set to be shorter than the height of the tip main body 11.

Further, the detailed configuration of the opening portions 12a to 12d is arbitrary, but in the embodiment, any one of the opening portions 12a to 12d is located at the discharge destination side or the suction destination side of the flow path device 20 (the right side in FIG. 4) in the contact state. Specifically, as illustrated in FIGS. 1A, 1B, and 4, the opening portion 12a is formed at the left side portion of the tip main body 11, the opening portion 12b is formed at the front side portion of the tip main body 11, the opening portion 12c is formed at the right side portion of the tip main body 11, and the opening portion 12d is formed at the rear side portion of the tip main body 11. Accordingly, the opening portion 12c can be located at the discharge destination side of the flow path device 20. Thus, since the test liquid L can be smoothly discharged to the flow path device 20 compared to a case in which the opening portion 12 is located only at a position other than the discharge destination side of the flow path device 20 in the contact state, it is possible to efficiently discharge the test liquid L.

(Configuration-Detailed Configuration of Tip-Shape Maintaining Portion)

Returning to FIGS. 1A, 1B, the shape maintaining portions 13a to 13d are shape maintaining members for maintaining the shape of the opening portions 12a to 12d in the contact state. The shape maintaining portions 13a to 13d are formed as a part of the tip main body 11. Specifically, as illustrated in FIGS. 1 and 4, the opening portions 12 are provided away from each other at the lower end portion of the tip main body 11 and the vicinity thereof.

Further, the shape and size of the shape maintaining portions 13a to 13d can be arbitrarily set as long as the shape of the opening portions 12a to 12d can be maintained in the contact state, but are set as below in the embodiment. That is, as illustrated in FIGS. 1A, 1B, and 4, the side surface shape of the shape maintaining portions 13a to 13d is set to be substantially rectangular. However, the present invention is not limited thereto, but, for example, a polygonal shape such as a triangular shape or a semi-circular shape may be used. Further, the width of the shape maintaining portions 13a to 13d is set to be shorter than the outer diameter of the tip main body 11 and the height of the shape maintaining portions 13a to 13d is set to be shorter than the height of the tip main body 11.

Further, the detailed configuration of the shape maintaining portions 13a to 13d is arbitrary, but in the embodiment, the shape maintaining portions 13a to 13d are in contact with the bottom surface portion 40a of the flow path device 20 to be described later by surface contact, line contact, or a plurality of point contacts in the contact state. Specifically, as illustrated in FIGS. 1A, 1B, and 4, each of the lower end portions of the shape maintaining portions 13a to 13d is formed in a flat surface shape so as to be in surface-contact with the bottom surface portion 40a of the flow path device 20 to be described later. Accordingly, the shape maintaining portions 13a to 13d can be brought into surface-contact with the bottom surface portion 40a of the flow path device 20 to be described later in the contact state and the shape of the opening portions 12a to 12d can be easily maintained in the contact state. Additionally, in this embodiment, the “bottom surface portion 40a” is a horizontal flat surface, but the shape of the bottom surface portion 40a is not limited thereto. Examples of other shapes will be described later.

(Configuration-Detailed Configuration of Tip-Others)

Further, the method of forming the tip 10 is arbitrary, but the tip main body 11, the shape maintaining portions 13a to 13d, and the opening portions 12a to 12d may be integrally formed by a method of injection-molding, for example, a resin material (or by a known glass molding method). Alternatively, the opening portions 12a to 12d may be formed by notching the tip main body 11 after integrally forming the tip main body 11 and the shape maintaining portions 13a to 13d with each other by injection-molding a resin material. Alternatively, the opening portions 12a to 12d may be formed by notching the existing tip main body 11.

The material of the tip 10 is not particularly limited, but a resin material such as polypropylene, polystyrene, polyethylene, polycarbonate, ABS resin, fluororesin (polytetrafluoroethylene (PTFE), perfluoroalkoxyalkane (PFA), perfluoroethylene propene polymer (FEP), ethylene tetrafluoroethylene copolymer (ETFE), polyvinylidene fluoride (PVDF), polychlorotrifluoroethylene (PCTFE), ethylene chlorotrifluoroethylene copolymer (ECTFE)), acrylic resin (polymethylmethacrylate (PMMA)), polyethylene terephthalate (PET), cyclic olefin resin (cycloolefin polymer (COP), cyclic olefin copolymer (COC)), polyacetal, and polyether ether ketone resin (PEEK resin), glass, metal, and the like can be used. Alternatively, an additive may be added in order to have various characteristics (conductiveness, antistatic property, radiation resistance, adsorption prevention of protein or nucleic acid, etc.). In particular, polypropylene which is easy to mold and has excellent heat resistance and chemical resistance can be preferably used. The tip 10 is preferably transparent or translucent so that the amount of the liquid can be visually recognized from the outside if the test liquid to be discharged and sucked has light resistance, but the tip may be non-transparent (black or the like) when the test liquid to be discharged and sucked has high photosensitivity.

A filter 14 may be provided inside the tip main body 11, particularly inside the vicinity of the upper open end 11b. Especially when using a nucleic acid amplification test, the DNA contained in the test liquid L may adhere to the discharge and suction member or float in the air to be mixed with the test liquid derived from another sample, and may cause a false positive. Therefore, it is preferable to provide a filter for preventing contamination. As the material and size of the filter 14, any filter used for the tip normally used in the nucleic acid amplification test can be used.

By the configuration of the above-described tip 10, the shape of the opening portions 12a to 12d can be maintained in the contact state by the shape maintaining portions 13a to 13d. Thus, it is possible to reliably discharge the test liquid L to the flow path device 20 in the contact state and to improve the user's convenience at the time of using the tip 10. Further, since the shape maintaining portions 13a to 13d are provided as a part of the tip main body 11, there is no need to separately provide a shape maintaining member. Accordingly, it is possible to decrease the number of members of the tip 10 and to improve the manufacturability of the tip 10.

(Configuration-Detailed Configuration of Flow Path Device)

Returning to FIGS. 2A, 2B, the configuration of the flow path device 20 will be described in detail below. However, the flow path device 20 can be manufactured in any shape, method, and material unless otherwise specified.

In the embodiment, the flow path device 20 includes, as illustrated in FIGS. 2A, 2B, 3A, 3B, 3C, and 4, the main body portion 30, a flow path 40, and the lid portion 50.

(Configuration-Detailed Configuration of Flow Path Device-Main Body Portion)

Returning to FIGS. 2A, 2B, the main body portion 30 is a basic structure of the flow path device 20. This main body portion 30 is formed as a substantially solid body (for example, a solid square body) and as illustrated in FIGS. 2A, 2B, 3A, 3B, 3C, and 4, the upper and lower surfaces of the main body portion 30 are disposed to be substantially in contact with the temperature controllers 60a and 60b. Here, the temperature controller 60 means to directly or indirectly heat and cool the flow path device 20 and any heating and cooling section used for ordinary real-time PCR reactions such as Peltier element, oil bath, and air can be used. In the example illustrated in the drawing, the temperature controller 60 is a Peltier element and is configured to directly heat and cool the flow path device 20.

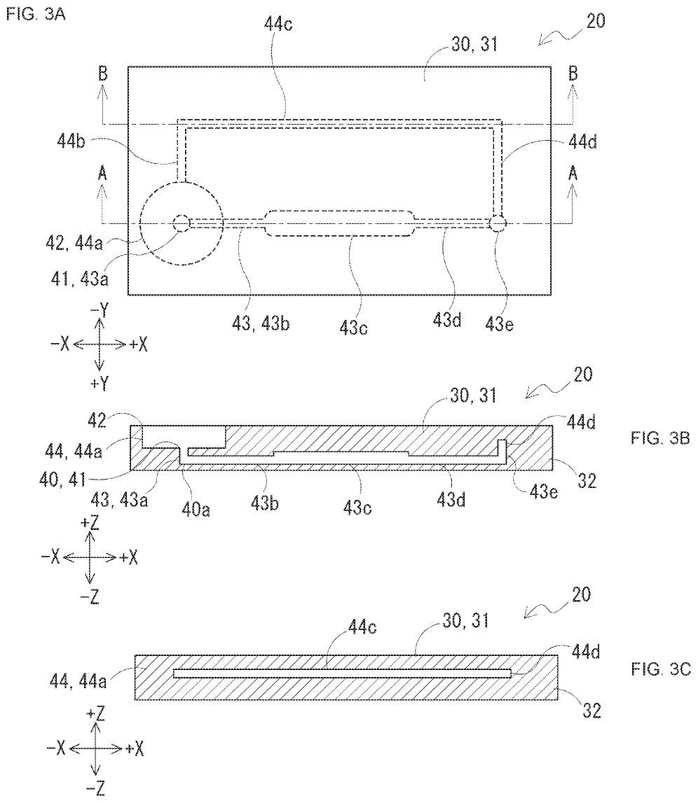

Further, the size of the main body portion 30 can be set as long as the flow path 40 can be accommodated, but is set as below in the embodiment. That is, as illustrated in FIGS. 2A, 2B, 3A, 3B, and 3C, the length of the main body portion 30 in the left and right direction is set to be longer than the length of the flow path 40 in the left and right direction, the length of the main body portion 30 in the front and rear direction is set to be longer than the length of the flow path 40 in the front and rear direction, and the length of the main body portion 30 in the up and down direction is set to be longer than the length of the flow path 40 in the up and down direction.

Further, the detailed configuration of the main body portion 30 is arbitrary, but in the embodiment, the main body portion includes an upper main body portion 31 and a lower main body portion 32. Among these, the upper main body portion 31 is a part of the basic structure of the main body portion 30 and constitutes the upper portion of the main body portion 30. Specifically, as illustrated in FIGS. 2A, 2B and 3A, 3B, 3C, the upper main body portion is formed to have a size in which a part of the flow path 40 (specifically, an outlet open end side portion 44 to be described later) is provided inside the upper main body portion 31. Further, the lower main body portion 32 is the other part of the basic structure of the main body portion 30 and constitutes the lower portion of the main body portion 30. Specifically, as illustrated in FIGS. 2A, 2B, 3A, 3B, 3C, the lower main body portion is formed to have a size in which the other part of the flow path 40 (specifically, an inlet open end side portion 43 to be described later) is provided inside the lower main body portion 32 and is disposed below the upper main body portion 31. Further, the method of forming the main body portion 30 is arbitrary, but in the embodiment, the main body portion is integrally formed by injection-molding a resin material (or by a 3D printer, cutting, etc.) as illustrated in FIGS. 2A, 2B, 3A, 3B, 3C. However, the present invention is not limited thereto and, for example, two or more separate members may be combined with each other.

The material of the flow path device 20 is not particularly limited, but when used for real-time PCR, it is preferable to use a translucent material (transparent material) in order to detect fluorescence. As such a material, a resin material such as polypropylene, polystyrene, polyethylene, polycarbonate, acrylic resin (polymethyl methacrylate (PMMA)), cyclic olefin resin (cycloolefin polymer (COP), cyclic olefin copolymer (COC)), silicone resin (PDMS), polyethylene terephthalate (PET), and photosensitive epoxy resin (SU-8), glass, and the like can be used. Furthermore, it is preferable to use a material with low autofluorescence. In particular, a cyclic olefin resin (cycloolefin polymer (COP), cyclic olefin copolymer (COC)), or quartz glass can be preferably used.

(Configuration-Detailed Configuration of Flow Path Device-Flow Path)

Returning to FIGS. 2A, 2B, the flow path 40 is for storing the test liquid L discharged from the tip 10 in the form of fluid. This flow path 40 is formed as a hollow portion (for example, a cylindrical hollow portion) and is provided inside the main body portion 30 as illustrated in FIGS. 2A, 2B, 3A, 3B, 3C.

Further, the detailed configuration of the flow path 40 is arbitrary, but in the embodiment, at least one or more inlet open ends 41 and at least one or more outlet open ends 42 in the plurality of different open ends overlap each other or are adjacent to each other and are exposed to the outside of the main body portion 30 of the flow path device 20. Specifically, as illustrated in FIGS. 2A, 2B, 3A, 3B, 3C, the flow path 40 includes the inlet open end 41, the outlet open end 42, the inlet open end side portion 43, and the outlet open end side portion 44. Note that, the “open end of the flow path 40” means the end portion of the flow path 40 in the axial direction. In the open end of the flow path 40, for example, the inlet open end 41 or the outlet open end 42 corresponds to the open end of the flow path 40 without the branch path and the inlet open end 41 or the outlet open end 42 of the branch path corresponds to the open end of the flow path 40 with the branch path in addition to the inlet open end 41 or the outlet open end 42 of the main flow path (which is a part not corresponding to the branch path) (however, the communication end communicating with the main flow path in the branch path is excluded). However, in the embodiment, the open ends of the flow path 40 without the branch path will be described.

(Configuration-Detailed Configuration of Flow Path Device-Flow Path-Inlet Open End, Outlet Open End)

The inlet open end 41 is an open end for allowing the test liquid L to flow into the flow path 40 in the open ends of the flow path 40 and is provided in the lower main body portion 32 as illustrated in FIGS. 2A, 2B, 3A, 3B, 3C. The outlet open end 42 is an open end for allowing the test liquid L inside the flow path 40 to flow to the outside of the flow path 40 in the open ends of the flow path 40 and is provided in the upper main body portion 31 as illustrated in FIGS. 2A, 2B, 3A, 3B, 3C. Here, the diameter of each of the inlet open end 41 and the outlet open end 42 is arbitrary, but in the embodiment, the diameter of the inlet open end 41 is set to be smaller than the diameter of the outlet open end 42. The present invention is not limited thereto and, for example, the diameter of the inlet open end 41 may be equal to or larger than the diameter of the outlet open end 42.

(Configuration-Detailed Configuration of Flow Path Device-Flow Path-Inlet Open End Side Portion)

Returning to FIGS. 2A, 2B, the inlet open end side portion 43 is a portion on the side of the inlet open end 41 in the portion of the flow path 40 and is a portion for storing the test liquid L. As illustrated in FIGS. 2A, 2B, 3A, 3B, 3C, this inlet open end side portion 43 is provided inside the lower main body portion 32 and includes the first inlet open end side portion 43a, a second inlet open end side portion 43b, a third inlet open end side portion 43c, a fourth inlet open end side portion 43d, and a fifth inlet open end side portion 43e.

Among these, the first inlet open end side portion 43a is disposed to project downward from the inlet open end 41 so that the axial direction of the first inlet open end side portion 43a follows the up and down direction. Further, the second inlet open end side portion 43b is disposed to project rightward from the first inlet open end side portion 43a so that the axial direction of the second inlet open end side portion 43b follows the left and right direction. Further, the third inlet open end side portion 43c is disposed to project rightward from the second inlet open end side portion 43b so that the axial direction of the third inlet open end side portion 43c follows the left and right direction. Further, the fourth inlet open end side portion 43d is disposed to project rightward from the third inlet open end side portion 43c so that the axial direction of the fourth inlet open end side portion 43d follows the left and right direction. Further, the fifth inlet open end side portion 43e is disposed to project upward from the fourth inlet open end side portion 43d so that the axial direction of the fifth inlet open end side portion 43e follows the up and down direction.

Further, the size of the inlet open end side portion 43 is arbitrary, but is set as below in the embodiment. That is, as illustrated in FIGS. 2A, 2B, 3A, 3B, 3C, the diameter of the first inlet open end side portion 43a is set to be substantially the same as the diameter of the inlet open end 41. Further, the length of the first inlet open end side portion 43a in the up and down direction is shorter than the length of the lower main body portion 32 in the up and down direction and is set to a length in which the side portion of the tip main body 11 can come into contact with the inlet open end 41 without a gap when the lower open end 11a of the tip main body 11 comes into contact with the bottom surface portion 40a of the first inlet open end side portion 43a (hereinafter, referred to as the “bottom surface portion 40a”). Further, the diameter of the second inlet open end side portion 43b is set to be smaller than the diameter of the inlet open end 41 and the length of the second inlet open end side portion 43b in the left and right direction is set to be shorter than the length of the lower main body portion 32 in the left and right direction. Further, the diameter of the third inlet open end side portion 43c is set to be larger than the diameter of the second inlet open end side portion 43b so that a desired amount of the test liquid L can be stored and the length of the third inlet open end side portion 43c in the left and right direction is also set to be longer than the length of the second inlet open end side portion 43b in the left and right direction. Further, the diameter of the fourth inlet open end side portion 43d is set to be substantially the same as the diameter of the second inlet open end side portion 43b and the length of the fourth inlet open end side portion 43d in the left and right direction is set to be substantially the same as (or slightly shorter than) the length of the second inlet open end side portion 43b in the left and right direction. Further, the diameter of the fifth inlet open end side portion 43e is set to be slightly larger than the diameter of the second inlet open end side portion 43b and the length of the fifth inlet open end side portion 43e in the up and down direction is set to be substantially the same as the length of the first inlet open end side portion 43a in the up and down direction.

(Configuration-Detailed Configuration of Flow Path Device-Flow Path-Outlet Open End Side Portion)

Returning to FIGS. 2A, 2B, the outlet open end side portion 44 is a portion on the side of the outlet open end 42 in the portion of the flow path 40 and can retain the vapor generated when the test liquid L stored in the inlet open end side portion 43 is heated by the temperature controller 60. The outlet open end and the outlet open end portion are portions which discharge internal air to the outside and introduce the test liquid L into the flow path when introducing the test liquid L from the inlet open end. As illustrated in FIGS. 2A, 2B, 3A, 3B, 3C, this outlet open end side portion 44 is provided inside the upper main body portion 31 and includes a first outlet open end side portion 44a, a second outlet open end side portion 44b, a third outlet open end side portion 44c, and a fourth outlet open end side portion 44d.

Among these, the first outlet open end side portion 44a is disposed to project downward from the outlet open end 42 so that the axial direction of the first outlet open end side portion 44a follows the up and down direction. Further, the second outlet open end side portion 44b is disposed to project backward from the first outlet open end side portion 44a so that the axial direction of the second outlet open end side portion 44b follows the front and rear direction. Further, the third outlet open end side portion 44c is disposed to project rightward from the second outlet open end side portion 44b so that the axial direction of the third outlet open end side portion 44c follows the left and right direction. Further, the fourth outlet open end side portion 44d is disposed to project forward from the second outlet open end side portion 44b so that the axial direction of the fourth outlet open end side portion 44d follows the front and rear direction.

Further, the diameter of the outlet open end side portion 44 is arbitrary, but is set as below in the embodiment. That is, as illustrated in FIGS. 2A, 2B, 3A, 3B, 3C, the diameter of the first outlet open end side portion 44a is set to be substantially the same as the diameter of the outlet open end 42 and the length of the first outlet open end side portion 44a in the up and down direction is set to be substantially the same as the length of the upper main body portion 31 in the up and down direction. Further, the diameter of the second outlet open end side portion 44b is set to be smaller than the diameter of the outlet open end 42 and the length of the second outlet open end side portion 44b in the front and rear direction is set to be shorter than the length of the upper main body portion 31 in the front and rear direction. Further, the diameter of the third outlet open end side portion 44c is set to be substantially the same as the diameter of the second outlet open end side portion 44b and the length of the third outlet open end side portion 44c in the left and right direction is set to be shorter than the length of the upper main body portion 31 in the left and right direction. Further, the diameter of the fourth outlet open end side portion 44d is set to be substantially the same as the diameter of the second outlet open end side portion 44b and the length of the third outlet open end side portion 44c in the front and rear direction is set to be shorter than the length of the upper main body portion 31 in the front and rear direction.

(Configuration-Detailed Configuration of Flow Path Device-Flow Path-Others)

Returning to FIGS. 2A, 2B, the detailed configuration of the inlet open end 41 and the outlet open end 42 is arbitrary, but in the embodiment, as illustrated in FIGS. 2A, 2B, 3A, 3B, 3C, the inlet open end 41 and the outlet open end 42 are arranged concentrically (specifically, concentrically when viewed from the plane direction) and are arranged not to be flush with each other (specifically, not to be flush with each other when viewed from the side). Specifically, the outlet open end 42 is disposed at the upper end portion of the upper main body portion 31 and the inlet open end 41 is disposed at the upper end portion of the lower main body portion 32 and is disposed to be flush with the lower end of the first outlet open end side portion 44a. Accordingly, since it is possible to decrease the area occupied by the inlet open ends 41 and the outlet open ends 42 in the main body portion 30 compared to a case in which at least one or more inlet open ends 41 and outlet open ends 42 are disposed adjacently to each other, it is possible to make the flow path 40 in a compact size. Further, since it is easy to suppress the test liquid L from flowing into and out from the flow path 40 through the outlet open end 42 when discharging the test liquid L by inserting the tip 10 through the flow path 40 through the inlet open end 41 compared to a case in which the inlet open end 41 and the outlet open end 42 are disposed to be flush with each other, it is possible to suppress the occurrence of contamination between the portions of the flow path 40 (or between the flow paths 40).

Further, the method of forming the main body portion 30 and the flow path 40 is arbitrary. However, the upper main body portion 31, the outlet open end 42, and the outlet open end side portion 44 may be integrally formed with each other and the lower main body portion 32, the inlet open end 41, and the inlet open end side portion 43 may be integrally formed with each other by injection-molding, for example, a resin material (or by a known glass molding method).

(Configuration-Detailed Configuration of Flow Path Device-Lid Portion)

Returning to FIGS. 2A, 2B, the lid portion 50 is for opening and closing at least one or more inlet open ends 41 and at least one or more outlet open ends 42 together. This lid portion 50 is configured by using, for example, a known lid for a flow path device (for example, a resinous truncated cone-shaped lid) or the like and is disposed to be inserted through the first outlet open end side portion 44a through the outlet open end 42 as illustrated in FIGS. 2A, 2B.

Further, the shape and size of the lid portion 50 are arbitrary, but are set as below in the embodiment. That is, as illustrated in FIGS. 2A, 2B, the side surface shape of the lid portion 50 is set to a substantially truncated cone shape in which the length of the upper end portion is larger than the length of the lower end portion. Further, the planar shape of the lid portion 50 is set to a substantially circular shape. Further, the diameter of the lid portion 50 is set to a size in which the entire lid portion 50 is in close contact with the outlet open end 42 when the lid portion 50 is inserted through the first outlet open end side portion 44a. For example, the minimum diameter of the lid portion 50 is set to be smaller than the outlet open end 42 and the maximum diameter of the lid portion 50 is set to be larger than the outlet open end 42. Further, the length of the lid portion 50 in the up and down direction is set to a size in which the lid portion 50 does not contact the first inlet open end side portion 43a (or the length may be set to a size that allows the contact) when the lid portion 50 is inserted through the first outlet open end side portion 44a. For example, the length may be substantially the same as (or longer or shorter than) the length of the first outlet open end side portion 44a in the up and down direction.

As the material forming the lid portion 50, those having excellent heat resistance and chemical resistance and having appropriate flexibility (or rigidity) and elasticity capable of sealing the outlet open end 42 can be preferably used. Specifically, silicone rubber, butyl rubber, nitrile rubber, natural rubber, synthetic natural rubber, butadiene rubber, styrene butadiene rubber, ethylene propylene rubber, chloroprene rubber, acrylic rubber, chlorosulfonated polyethylene rubber, urethane rubber, fluororubber, and the like can be mentioned.

By the above-described configuration of the flow path device 20, at least one or more inlet open ends 41 and outlet open ends 42 can be opened and closed together by one lid portion 50 and the work of opening and closing the inlet open end 41 and the outlet open end 42 can be simplified compared to a case in which the inlet open end 41 and the outlet open end 42 disposed to be away from each other are individually opened and closed by using each dedicated lid portion. Thus, it is effective in constructing a system for automating the opening and closing work of opening and closing the inlet open end 41 and the outlet open end 42.

(Test Method)

Next, a specimen test method which is performed by the biological component test system 1 of the present invention will be described. The test method of the embodiment includes an injection step, an amplification step, and a detection step. Additionally, as a premise of this test method, the flow path device 20 will be described as one horizontally installed while being in contact with the temperature controller 60.

(Test Method-Injection Step)

First, an injection step will be described. The injection step is a step of injecting the test liquid L into the flow path device 20. The test liquid L exemplified in this embodiment is a real-time PCR test liquid obtained by mixing a treated sample, containing DNA obtained by treating a biological sample, with DNA polymerase, dNTP, primer, and TaqMan (trademark) probe, but the test liquid in the present invention is not limited thereto.

Specifically, the test liquid is sucked to the tip 10 attached to the nozzle of the discharge and suction member, the nozzle and the tip 10 are moved to the upper side of the inlet open end 41 of the flow path device 20, and the tip 10 is moved downward and is inserted through the first inlet open end side portion 43a through the inlet open end 41. In this case, the lower end portion of the tip 10 is in contact with or near the bottom surface portion 40a of the flow path device 20, but even in the contact state, the shape of the opening portions 12a to 12d is maintained by the shape maintaining portions 13a to 13d. Next, a predetermined amount of the test liquid L is discharged from the nozzle of the discharge and suction member and the test liquid L is injected into the flow path 40 through the tip 10. Here, the predetermined amount is arbitrary, but may be set to, for example, a degree in which a desired amount of the test liquid L is stored in the third inlet open end side portion 43c and the test liquid L does not flow into the outlet open end side portion 44. Next, the tip 10 is detached from the flow path device 20 by moving the nozzle of the discharge and suction member upward and then to the left or right.

(Test Method-Amplification Step, Detection Step)

Next, an amplification step and a detection step will be described. The amplification step is a step of amplifying the target sequence of DNA (or RNA) contained in the test liquid L contained in the flow path 40 after the injection step. The detection step is a step of detecting the target sequence of the amplified DNA after or during the amplification step. As described above, in the embodiment, the amplification step and the detection step are performed in parallel in order to perform the nucleic acid amplification test based on the real-time PCR method (intercalation method or fluorescently labeled probe hybridization method).

Specifically, the lid portion 50 is inserted through the first outlet open end side portion 44a through the outlet open end 42 of the flow path device 20 by using the attachment and detachment portion. Accordingly, the outlet open end 42 and the inlet open end 41 are closed together by the lid portion 50. Next, the temperature control of the test liquid L stored in the flow path device 20 (particularly, the test liquid L inside the third inlet open end side portion 43c) and the detection of fluorescence generated by the amplification of DNA are performed by operating the temperature controller 60 and the detector. Nucleic acid amplification is performed by controlling the temperature in a cycle of denaturation temperature (about 95° C.)-annealing temperature (about 50 to 75° C.)-elongation temperature (about 60 to 75° C.). The detailed denaturation, annealing, and elongation temperature and the retention time of each temperature are adjusted and set in advance according to the template DNA, primer sequence, and target sequence. The temperature cycle is usually performed about 25 to 50 times.

(Discarding Step)

After the detection step, the test liquid in the flow path device can be used for later analysis, but is preferably discarded for each flow path device without opening the lid portion. If the lid portion is removed and the flow path is opened to the outside, there is a risk that a large amount of nucleic acid after the amplification reaction will diffuse into the environment in the state of an aerosol or the like and will contaminate other unreacted test liquids, but the risk can be avoided by discarding the test liquid without opening the lid portion. In particular, in a nucleic acid amplification test in which a target sequence of DNA is amplified several million times, it can be said that the effect of avoiding contamination risk is great.

According to the above-described test method, the biological component test system 1 can automatically perform the nucleic acid amplification test and hence the nucleic acid amplification test can be easily performed.

### Effect of Embodiment

In this way, according to the embodiment, there are provided at least one or more flow paths 40 provided inside the main body portion 30, one or more flow paths 40 being configured so that at least one or more inlet open ends 41 and at least one or more outlet open ends 42 in the plurality of different open ends in the at least one or more flow paths 40 overlap each other or are adjacent to each other and are exposed to the outside of the main body portion 30, and the lid portion 50 opening and closing at least one or more inlet open ends 41 and at least one or more outlet open ends 42 together. Accordingly, since at least one or more inlet open ends 41 and outlet open ends 42 can be opened and closed together by one lid portion 50, it is possible to simplify the work of opening and closing the inlet open end 41 and the outlet open end 42 compared to a case in which the inlet open end 41 and the outlet open end 42 disposed to be away from each other are individually opened and closed by using a seal member covering one surface of the main body portion or using a plurality of lid portions and to more easily perform the position control or the like when the work is automated.

Further, since at least one or more inlet open ends 41 and at least one or more outlet open ends 42 are arranged concentrically, it is possible to decrease the area occupied by the inlet open ends 41 and the outlet open ends 42 in the main body portion 30 compared to a case in which at least one or more inlet open ends 41 and outlet open ends 42 are arranged adjacently to each other and to make the flow path 40 in a compact size.

Further, since at least one or more inlet open ends 41 and at least one or more outlet open ends 42 are arranged not to be flush with each other, it is easy to suppress the test liquid L from flowing into and out from the flow path 40 through the outlet open end 42 when discharging the test liquid L by inserting the tip 10 through the flow path 40 through the inlet open end 41 compared to a case in which the inlet open end 41 and the outlet open end 42 are arranged to be flush with each other. Accordingly, it is possible to suppress the occurrence of contamination between the portions of the flow path 40 (or between the flow paths 40).

### [III] Modified Example of Embodiment

As described above, the embodiments of the present invention have been described, but the specific configuration and means of the present invention can be arbitrarily modified and improved within the scope of the technical idea of each invention described in the claims. Hereinafter, such modified examples will be described.

(Problems to be Solved by the Invention and Effects of the Invention)

First, the problems to be solved by the invention and the effect of the invention are not limited to the above-described contents and the present invention can solve a problem not described above or have an effect not described above. Further, only some of the problems described may be solved or only some of the effects described may be achieved.

(Shape, Numerical Value, Structure, and Time Series)

In the components illustrated in the embodiments and drawings, the shape, numerical value, structure of a plurality of components, or the correlation of time series can be arbitrarily modified and improved within the scope of the technical idea of the present invention.

(Biological Component Test System)

In the above-described embodiment, a case has been described in which the biological component test system 1 includes the discharge and suction member and the attachment and detachment portion, but the present invention is not limited thereto. For example, at least one of the discharge and suction member and the attachment and detachment portion may be omitted. In this case, the work of discharging and sucking the test liquid L or the work of attaching or detaching the lid portion 50 may be performed manually. Alternatively, the discharge and suction member and the attachment and detachment portion may be integrated with each other.

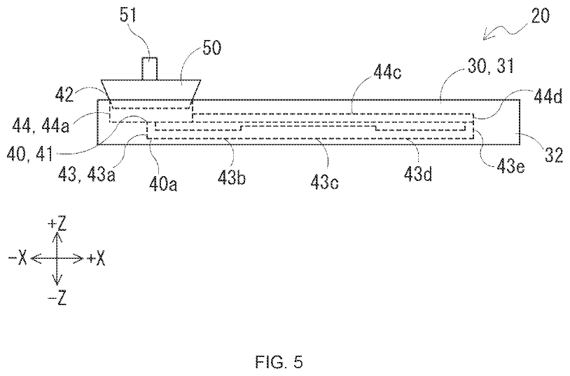

As illustrated in FIG. 5, the upper end portion of the lid portion 50 may be provided with a grip portion 51 for gripping the lid portion 50 (for example, an annular (or cylindrical) grip portion 51).

(Tip)

In the above-described embodiment, a case has been described in which the tip 10 includes the opening portion 12 and the shape maintaining portion 13, but the present invention is not limited thereto. For example, the opening portion 12 and the shape maintaining portion 13 may be omitted.

Further, in the above-described embodiment, a case has been described in which the tip 10 discharges the test liquid L to the flow path device 20, but the present invention is not limited thereto. For example, the test liquid L may be sucked to the flow path device 20 or the test liquid L may be discharged and sucked to the flow path device 20.

Further, in the above-described embodiment, a case has been described in which the tip main body 11 is a cylindrical body, but the present invention is not limited thereto. For example, a cylindrical body having a polygonal annular shape (for example, a triangular annular shape) in cross section may be used.

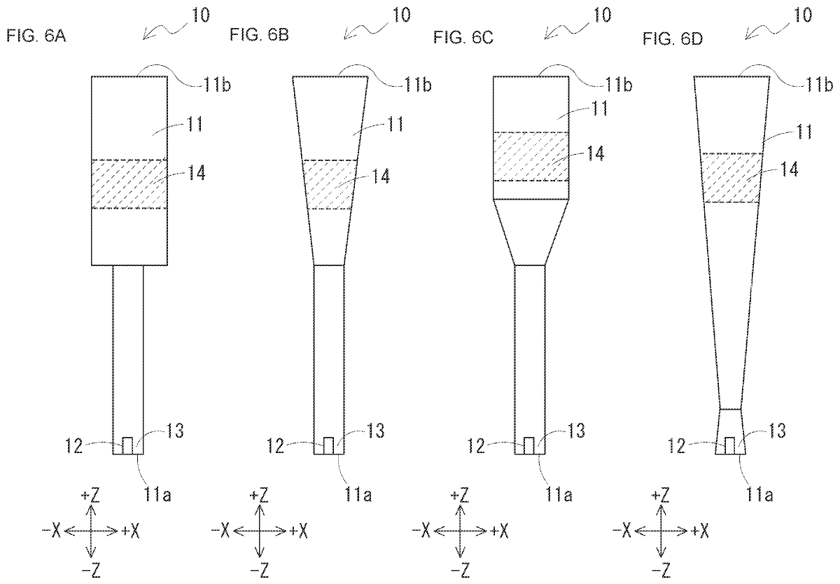

Further, in the above-described embodiment, a case has been described in which the side surface shape of the tip main body 11 is tapered, but the present invention is not limited thereto. For example, the tip main body 11 may have a rectangular shape in which the entire outer diameter is constant. Alternatively, as illustrated in FIGS. 6A, 6B, 6C, 6D, the tip main body 11 may have a shape in which the outer diameter is not constant.

(Opening Portion)

In the above-described embodiment, a case has been described in which the number of the installed opening portions 12 is four, but the present invention is not limited thereto. For example, the number may be smaller than four or five or more. In this case, the number of the installed shape maintaining portions 13 is changed according to the number of the installed opening portions 12.

Further, in the above-described embodiment, a case has been described in which the shape and size of the opening portions 12a to 12d is set so that the total discharge amount of the test liquid L of the opening portions 12a to 12d is substantially the same as the discharge amount of the test liquid L of the lower open end 11a, but the present invention is not limited thereto. For example, the total discharge amount of the test liquid L of the opening portions 12a to 12d may be set to be larger (or smaller) than the discharge amount of the test liquid L of the lower open end 11a.

Further, in the above-described embodiment, a case has been described in which any one of the opening portions 12a to 12d in the contact state is located at the discharge destination side of the flow path device 20, but the present invention is not limited thereto. For example, the opening portion may be located only at a position other than the discharge destination side of the flow path device 20.

(Shape Maintaining Portion)

In the above-described embodiment, a case has been described in which the number of the installed shape maintaining portions 13 is four, but the present invention is not limited thereto. For example, the number may be smaller than four or five or more.

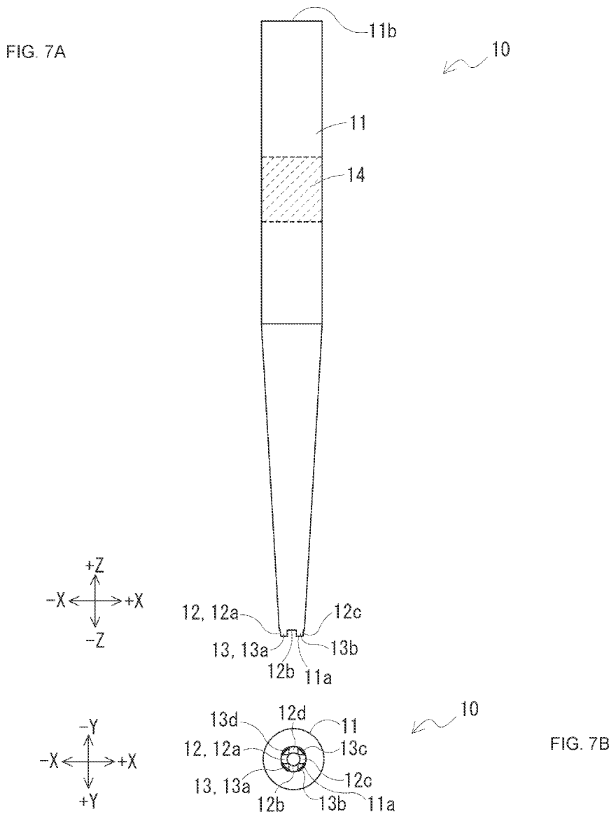

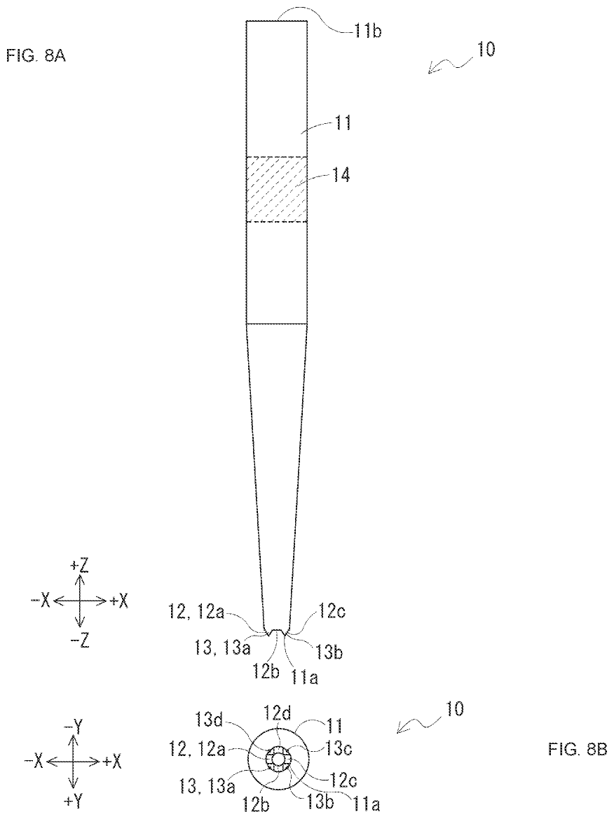

Further, in the above-described embodiment, a case has been described in which the lower end portion of each of the shape maintaining portions 13a to 13d is formed in a flat surface shape to come into surface-contact with the bottom surface portion 40a of the flow path device 20 in the contact state, but the present invention is not limited thereto. For example, as illustrated in FIGS. 7A, 7B, the lower end portion of each of the shape maintaining portions 13a to 13d may be formed in an inclined surface shape so that only the outer edge portion of the lower end portion of each of the shape maintaining portions 13a to 13d comes into line-contact with the bottom surface portion 40a of the flow path device 20. Alternatively, as illustrated in FIGS. 8A, 8B, the shape maintaining portions 13a to 13d may be formed in a pen tip shape that becomes sharper downward so that the lower end portion of each of the shape maintaining portions 13a to 13d comes into point-contact with the bottom surface portion 40a of the flow path device 20 (that is, all the shape maintaining portions 13a to 13d come into contact with the bottom surface portion 40a of the flow path device 20 at a plurality of points).

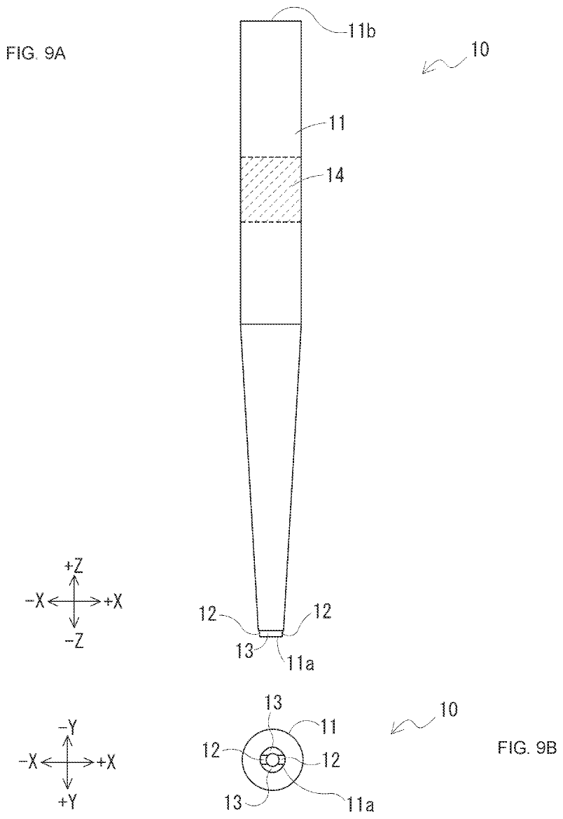

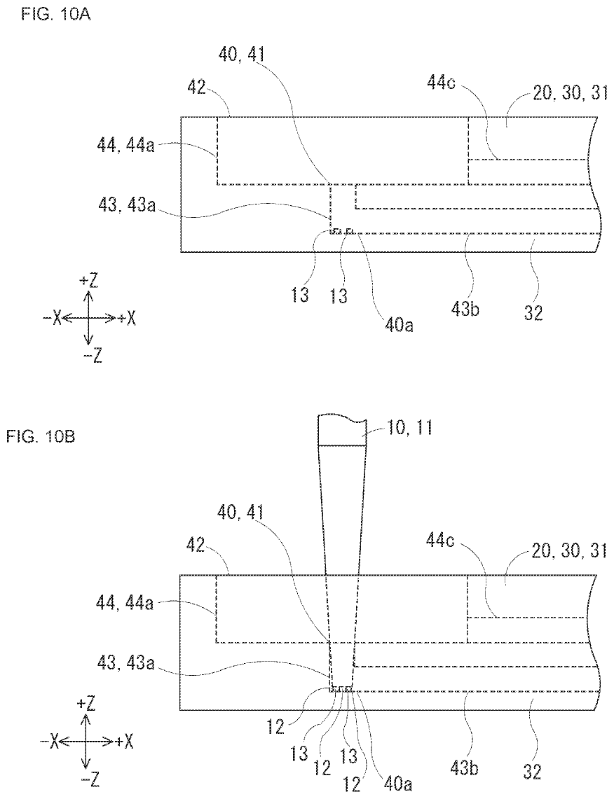

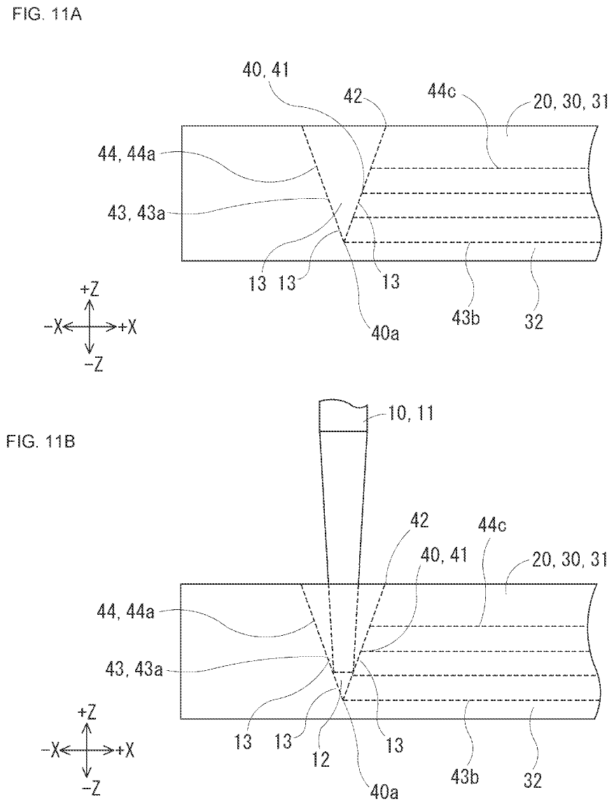

Further, in the above-described embodiment, a case has been described in which the shape maintaining portion 13 is configured as a part of the tip main body 11, but the present invention is not limited thereto. For example, as illustrated in FIGS. 9A, 9B, the shape maintaining portion 13 may be attached to the lower end portion of the tip main body 11 and may be configured as a member separated from the tip main body 11. In this case, the method of forming the tip 10 is arbitrary, but the tip main body 11, the shape maintaining portion 13, and the opening portion 12 may be integrally formed with each other by injection-molding, for example, a resin material. Alternatively, as illustrated in FIGS. 10A, 10B, the shape maintaining portion 13 may be attached to the bottom surface portion 40a of the flow path device 20 and may be formed as a member separated from the tip main body 11 (for example, a plurality of shape maintaining portions may be provided to be away from each other in the entire length of the first inlet open end side portion 43a in the front and rear direction). Alternatively, a portion corresponding to the bottom surface portion 40a may be formed as a shape other than the flat surface and may serve as the shape maintaining portion 13. For example, in FIGS. 11A, 11B, the shape maintaining portion 13 may be configured as a part of the lower main body portion 32 of the flow path device 20 (for example, a portion corresponding to the first inlet open end side portion 43a) and may be configured as a member separated from the tip main body 11 (for example, the diameter of the first inlet open end side portion 43a becomes smaller downward and the diameter of the portion slightly above the lower end of the first inlet open end side portion 43a becomes smaller than the outer diameter of the lower open end 11a). When the shape maintaining portion 13 according to FIGS. 10A, 10B, 11A, 11B is provided, as illustrated in FIGS. 10A, 10B, 11A, 11B, the opening portion 12 is provided between the lower end portion of the tip main body 11 and the bottom surface portion 40a of the flow path device 20 in the contact state. Further, the method of forming the flow path device 20 according to FIGS. 10A, 10B, 11A, 11B is arbitrary, but the main body portion 30, the flow path 40, the tip main body 11, and the shape maintaining portion 13 may be integrally formed with each other by injection-molding, for example, a resin material. Accordingly, the shape of the opening portion 12 in the contact state can be maintained by the shape maintaining portion 13. Thus, it is possible to reliably discharge or suck the test liquid L to or from the flow path device 20 in the contact state and to improve the user's convenience at the time of using the biological component test system 1.

(Flow Path Device)

In the above-described embodiment, a case has been described in which the flow path device 20 is used for the nucleic acid amplification test, but the present invention is not limited thereto. For example, the flow path device may be used in a genetic test or a serum test to contain a test liquid or a reagent so as not to dry or evaporate.

Further, in the above-described embodiment, a case has been described in which the main body portion 30 is formed as a solid rectangular body, but the present invention is not limited thereto. For example, the main body portion may be formed in a shape other than the solid rectangular body (for example, a polygonal prism such as a solid hexagonal prism, a solid cylinder, or the like).

Further, in the above-described embodiment, the main body portion 30 is integrally formed, but the present invention is not limited thereto. For example, two or more separated members divided in the up and down direction or the left and right direction may be combined and integrated with each other by adhering, welding, or the like.

(Flow Path)

In the above-described embodiment, a case has been described in which the number of the installed flow paths 40 is one, but the present invention is not limited thereto. For example, the number may be two or more. In this case, the inlet open end 41 and the outlet open end 42 may be disposed to overlap or be adjacent to each other so that the inlet open ends 41 and the outlet open ends 42 of the plurality of flow paths 40 are opened and closed together by the lid portion 50.

Further, in the above-described embodiment, a case has been described in which the flow path 40 includes each of the inlet open end 41 and the outlet open end 42, but the present invention is not limited thereto. For example, when the flow path 40 is provided with at least one or more branch paths, at least one of the inlet open end 41 and the outlet open end 42 may be provided at a plurality of positions. In this case, the plurality of inlet open ends 41 and outlet open ends 42 may be disposed to overlap or be adjacent to each other so that the plurality of inlet open ends 41 and outlet open ends 42 are opened and closed together by the lid portion 50.

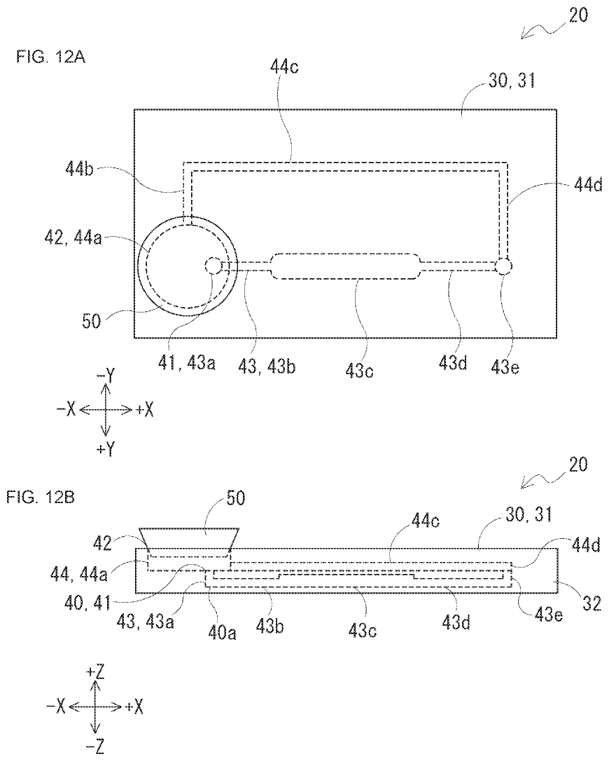

Further, in the above-described embodiment, a case has been described in which the inlet open end 41 and the outlet open end 42 are arranged concentrically, but the present invention is not limited thereto. These open ends may be arranged non-concentrically. For example, as illustrated in FIGS. 12A, 12B, the inlet open end 41 and the outlet open end 42 may be disposed to overlap each other. Alternatively, as illustrated in, the inlet open end 41 and the outlet open end 42 may be disposed adjacent to each other. In this case, the inlet open end 41 and the outlet open end 42 may be disposed at the upper end portion of the upper main body portion 31 (that is, these may be disposed to be flush with each other when viewed from the side). Further, the length of the boundary portion between the inlet open end 41 and the outlet open end 42 in the up and down direction may be set to a height in which a part of the lid portion 50 can be inserted through the first inlet open end side portion 43a and the first outlet open end side portion 44a.

Further, in the above-described embodiment, a case has been described in which the outlet open end side portion 44 is provided at the upper main body portion 31 and the inlet open end side portion 43 is provided at the lower main body portion 32, but the present invention is not limited thereto. For example, the inlet open end side portion 43 may be provided at the upper main body portion 31 and the outlet open end side portion 44 may be provided at the lower main body portion 32. In this case, the inlet open end 41 may be disposed at the upper end portion of the upper main body portion 31 and the outlet open end 42 may be disposed at the upper end portion of the lower main body portion 32 to be flush with the lower end of the first inlet open end side portion 43a.

Further, in the above-described embodiment, a case has been described in which the diameter of each of the first inlet open end side portion 43a and the first outlet open end side portion 44a is set to be constant, but the present invention is not limited thereto. For example, as illustrated in FIGS. 11A, 11B, the diameter of the first inlet open end side portion 43e may be set to be smaller as it goes away from the inlet open end 41. In this case, it is preferable that the diameter of the first inlet open end side portion 43a becomes smaller downward and the diameter of the portion slightly above the lower end of the first inlet open end side portion 43a becomes smaller than the outer diameter of the lower open end 11a of the tip 10. In particular, it is preferable to have a structure in which all annular lines constituting the lower open end 11a of the tip 10 are in contact with the wall portion of the first inlet open end side portion 43a. When the entire open end is in contact with the wall portion, the second inlet open end side portion 43b is substantially sealed by the tip 10. When the test liquid L is discharged in this state, an appropriate pressure is applied to the test liquid L so that the test liquid L can be efficiently introduced into the flow path. On the other hand, when the diameter of the inlet open end 41 is set to be larger than the lower portion, it is possible to insert the tip 10 without accurately aligning the tip 10. Even when there is a slight deviation between the center position of the diameter of the tip 10 and a predetermined position when inserting the tip 10, the tip 10 is inserted until the tip comes into contact with the first inlet open end side portion 43a so that the tip can come into contact with the first inlet open end side portion 43a at a predetermined position due to the flexibility of the tip material.

(Lid Portion)

In the above-described embodiment, a case has been described in which the diameter of the lid portion 50 is set to a size in which the entire lid portion 50 is in close contact with the outlet open end 42 when the lid portion 50 is inserted through the first outlet open end side portion 44a, but the present invention is not limited thereto. For example, the diameter may be set to a size in which only a part of the lid portion 50 is in close contact with the outlet open end 42.

Further, in the above-described embodiment, a case has been described in which the lid portion 50 is formed as a resinous cylindrical body, but the present invention is not limited thereto. For example, the lid portion may be formed as a flat thin film (for example, a resin seal or the like).

One embodiment of the present invention provides a biological component test flow path device for storing a substance used to test a biological component in a specimen separated from a living body, the biological component test flow path device comprises: a main body portion; at least one or more flow paths which are provided inside the main body portion and are provided so that at least one or more inlet open ends and at least one or more outlet open ends in a plurality of different open ends of the at least one or more flow paths overlap each other or are adjacent to each other and are exposed to the outside of the main body portion; and a lid portion which opens and closes the at least one or more inlet open ends and the at least one or more outlet open ends together.

According to this embodiment, there are provided at least one or more flow paths which are provided inside the main body portion and are provided so that at least one or more inlet open ends and at least one or more outlet open ends in a plurality of different open ends of the at least one or more flow paths overlap each other or are adjacent to each other and are exposed to the outside of the main body portion; and a lid portion which opens and closes the at least one or more inlet open ends and the at least one or more outlet open ends together. Accordingly, since at least one or more inlet open ends and outlet open ends can be opened and closed together by one lid portion, it is possible to simplify the work of opening and closing the inlet open end and the outlet open end compared to a case in which the inlet open end and the outlet open end disposed to be away from each other are individually opened and closed by using a seal member covering one surface of the main body portion or using a plurality of lid portions and to more easily perform the position control or the like when the work is automated.

Another embodiment of the present invention provides the biological component test flow path device according to the above embodiment, wherein the at least one or more inlet open ends and the at least one or more outlet open ends are arranged concentrically.

According to this embodiment, since the at least one or more inlet open ends and the at least one or more outlet open ends are arranged concentrically, it is possible to decrease the area occupied by the inlet open ends and the outlet open ends in the main body portion compared to a case in which at least one or more inlet open ends and outlet open ends are arranged adjacently to each other and to make the flow path in a compact size.

Another embodiment of the present invention provides the biological component test flow path device according to the above embodiment, wherein the at least one or more inlet open ends and the at least one or more outlet open ends are arranged not to be flush with each other.

According to this embodiment, since the at least one or more inlet open ends and the at least one or more outlet open ends are arranged not to be flush with each other, it is easy to suppress the substance from flowing into and out from the flow path through the outlet open end when discharging the substance by inserting a tip through the flow path through the inlet open end compared to a case in which the inlet open end and the outlet open end are arranged to be flush with each other. Accordingly, it is possible to suppress the occurrence of contamination between the portions of the flow path (or between the flow paths).

Another embodiment of the present invention provides the biological component test flow path device according to the above embodiment, wherein a diameter of a portion which projects from the inlet open end or the outlet open end toward a predetermined direction and corresponds to a part of a portion on the inlet open end side or the outlet open end side in portions of the at least one or more flow paths becomes smaller as it goes away from the inlet open end side or the outlet open end side.

According to this embodiment, since a diameter of a portion which projects from the inlet open end or the outlet open end toward a predetermined direction and corresponds to a part of a portion on the inlet open end side or the outlet open end side in portions of the at least one or more flow paths becomes smaller as it goes away from the inlet open end side or the outlet open end side. Accordingly, when injecting a test liquid with a microchip, needle, etc., from the inlet open end, the smallest diameter of the above diameters is smaller than the smallest diameter of the microchip, needle, etc., so that the discharge and suction openings of the microchip, needle, etc., are not blocked by the device material.

Another embodiment of the present invention provides the biological component test flow path device according to the above embodiment, comprises: the biological component test flow path device according to any one of the above embodiments.

According to this embodiment, there are provided at least one or more flow paths which are provided inside the main body portion and are provided so that at least one or more inlet open ends and at least one or more outlet open ends in a plurality of different open ends of the at least one or more flow paths overlap each other or are adjacent to each other and are exposed to the outside of the main body portion; and a lid portion which opens and closes the at least one or more inlet open ends and the at least one or more outlet open ends together. Accordingly, since at least one or more inlet open ends and outlet open ends can be opened and closed together by one lid portion, it is possible to simplify the work of opening and closing the inlet open end and the outlet open end compared to a case in which the inlet open end and the outlet open end disposed to be away from each other are individually opened and closed by using a seal member covering one surface of the main body portion or using a plurality of lid portions and to more easily perform the position control or the like when the work is automated.

Another embodiment of the present invention provides a tip for testing biological components for discharging or suction a substance used for testing biological components in a sample separated from a living body into a container, comprises: a tip main body having cylindrical shape, at least one or more openings that are connected to a discharge and suction side open end which is an open end of the tip main body where the substance is discharged or sucked, and that are for allowing the substance to flow in to and flow out from a side of the tip main body when discharging or aspirating the substance in a contact state in which an end portion on a discharge side or a suction side of the substance in end portions of the tip for testing biological components is in contact with a bottom surface portion of the container, and a shape maintaining unit for maintaining the shape of the at least one or more openings in the contact state.

According to this embodiment, there are provided at least one or more openings that are connected to a discharge and suction side open end which is an open end of the tip main body where the test fluid (the substance) is discharged or sucked, and that are for allowing the substance to flow in to and flow out from a side of the tip main body when discharging or aspirating the substance in a contact state in which an end portion on a discharge side or a suction side of the substance in end portions of the tip for testing biological components is in contact with a bottom surface portion of the container, and a shape maintaining unit for maintaining the shape of the at least one or more openings in the contact state. Accordingly, the shape of the opening portion in the contact state can be maintained by the shape maintaining unit. Thus, it is possible to reliably discharge or suck the test liquid to or from the container in the contact state and to improve the user's convenience at the time of using the tip for testing biological components.

Another embodiment of the present invention provides the tip for testing biological components according to the above embodiment, wherein the shape maintaining unit is configured to be capable of contacting with the bottom surface portion of the container by surface contact, line contact, or a plurality of point contacts in the contact state.

According to this embodiment, since the shape maintaining unit is configured to be capable of contacting with the bottom surface portion of the container by surface contact, line contact, or a plurality of point contacts in the contact state, the shape maintaining unit can be brought into contact with the bottom surface portion of the container by surface contact, line contact, or a plurality of point contacts in the contact state, the shape of the opening portions can be easily maintained in the contact state.

Another embodiment of the present invention provides the tip for testing biological components according to the above embodiment, wherein the openings are configured to be located at the discharge side or the suction side of the container.

According to this embodiment, since the openings are configured to be located at the discharge side or the suction side of the container in the contact state, the opening portion can be located at the discharge side or the suction side of the container. Thus, the test liquid can be smoothly discharged to or sucked from the container compared to a case in which the opening portion is located only at a position other than the discharge destination side or the suction side of the container in the contact state, it is possible to efficiently discharge or suck the test liquid.

Another embodiment of the present invention provides the tip for testing biological components according to the above embodiment, wherein the shape maintaining unit is formed as a part of the tip main body.

According to this embodiment, since the shape maintaining unit is formed as a part of the tip main body, there is no need to separately provide a shape maintaining member. Accordingly, it is possible to decrease the number of members of the tip for testing biological components and to improve the manufacturability of the tip for testing biological components.

Another embodiment of the present invention provides a system used for testing biological components in a sample separated from a living body, the system for testing biological components comprising a container, and a tip for discharging or aspirating a substance used for testing the biological components into a container, the system comprises: the tip with a tip main body having cylindrical shape, at least one or more openings that are connected to a discharge and suction side open end which is an open end of the tip main body where the substance is discharged or sucked, and that are for allowing the substance to flow in to and flow out from a side of the tip main body when discharging or aspirating the substance in a contact state in which an end portion on a discharge side or a suction side of the substance in end portions of the tip for testing biological components is in contact with a bottom surface portion of the container, and a shape maintaining unit for maintaining the shape of the at least one or more openings in the contact state.

According to this embodiment, there are provided at least one or more openings that are connected to a discharge and suction side open end which is an open end of the tip main body where the substance is discharged or sucked, and that are for allowing the substance to flow in to and flow out from a side of the tip main body when discharging or aspirating the substance in a contact state in which an end portion on a discharge side or a suction side of the substance in end portions of the tip for testing biological components is in contact with a bottom surface portion of the container, and a shape maintaining unit for maintaining the shape of the at least one or more openings in the contact state. Accordingly, the shape of the opening portion in the contact state can be maintained by the shape maintaining unit. Thus, it is possible to reliably discharge or suck the test liquid to or from the container in the contact state and to improve the user's convenience at the time of using the system for testing biological components.

### REFERENCE SIGNS LIST

1 Biological component test system

10 Tip

11 Tip main body

11a Lower open end

11b Upper open end

12, 12a to 12d Opening portion

13, 13a to 13d Shape maintaining portion

14 Filter

20 Flow path device

30 Main body portion

31 Upper main body portion

32 Lower main body portion

40 Flow path

40a Bottom surface portion

41 Inlet open end

42 Outlet open end

43 Inlet open end side portion

43a First inlet open end side portion

43b Second inlet open end side portion

43c Third inlet open end side portion

43d Fourth inlet open end side portion

43e Fifth inlet open end side portion

44 Outlet open end side portion

44a First outlet open end side portion

44b Second outlet open end side portion

44c Third outlet open end side portion

44d Fourth outlet open end side portion

50 Lid portion

51 Grip portion

60, 60a, 60b Temperature controller

L Test liquid

1. A biological component test flow path device for storing a substance used to test a biological component in a specimen separated from a living body, the biological component test flow path device comprising:
a main body portion;
at least one or more flow paths which are provided inside the main body portion and are provided so that at least one or more inlet open ends and at least one or more outlet open ends in a plurality of different open ends of the at least one or more flow paths overlap each other or are adjacent to each other and are exposed to the outside of the main body portion; and
a lid portion which opens and closes the at least one or more inlet open ends and the at least one or more outlet open ends together.

2. The biological component test flow path device according to claim 1,
wherein the at least one or more inlet open ends and the at least one or more outlet open ends are arranged concentrically.

3. The biological component test flow path device according to claim 1,
wherein the at least one or more inlet open ends and the at least one or more outlet open ends are arranged not to be flush with each other.

4. The biological component test flow path device according to claim 1,
wherein a diameter of a portion which projects from the inlet open end or the outlet open end toward a predetermined direction and corresponds to a part of a portion on the inlet open end side or the outlet open end side in portions of the at least one or more flow paths becomes smaller as it goes away from the inlet open end side or the outlet open end side.

5. A biological component test system comprising:
the biological component test flow path device according to claim 1.
